(19) United States
(12) Patent Application Publication
(10) Pub. No.: US 2013/0245395 A1
(43) Pub. Date: Sep. 19, 2013

(54) Pillow and Mattress for Reducing Snoring and Sleep Apnea

(71) Applicant: Armin Bidarian Moniri, Goteborg (SE)

(72) Inventor: Armin Bidarian Moniri, Goteborg (SE)

(21) Appl. No.: 13/881,208

(22) PCT Filed: Oct. 25, 2011
(86) PCT No.: PCT/SE2011/051268

§ 371 Date: May 21, 2013

Related U.S. Application Data
(60) Provisional application No. 61/455,694, filed on Oct. 25, 2010.

(51) Int. Cl. A47G 9/10; A61B 5/00; A61B 7/00; A61B 5/11; A61B 5/01

(52) U.S. Cl. CPC A47G 9/1054; A61B 5/11; A61B 5/01; A61B 7/00; A61B 5/4818; A61B 5/6891
USPC 600/301; 5/636; 5/640; 600/595; 600/549; 600/586

(57) ABSTRACT

The present invention provides an improved pillow and mattress set to be used as a means for reducing snoring and obstructive sleep apnea during sleep by allowing a true prone position of the head. The pillow and mattress set further provides a support for lying with the body in a prone position. The pillow and mattress set comprises a pillow to support the head in a prone position and a mattress to support the body in a prone position, wherein the pillow and mattress are freely adjustable in relation to each other.

### FIELD OF THE INVENTION

The present invention relates generally to a sleep pillow and mattress set. More specifically the invention relates to a set of sleep pillow and mattress that allows for sleeping in a comfortable prone position that makes the airways of the user remain open during sleep and for reduction of snoring and sleep apnea, including a pillow and a mattress for use as a head support, especially for support of the head of a human in a prone position during sleep, the pillow having top and bottom surfaces and said mattress having top and bottom surfaces and two short ends.

### BACKGROUND OF THE INVENTION

Snoring is produced by vibration of the soft tissue in the upper airways during sleep. Reduction in the transverse diameter of the airways during sleep and the soft tissue relaxation are the primary causes of this condition. Some snorers can develop an obstruction of the upper airways with partial (hypopnea) or total (apnea) cessation of breathing. When more than five to ten hypopneas and/or apneas occur per hour of sleep the patient is considered to have obstructive sleep apnea (OSA).

Previous observations confirm the fact that many mammals snore during sleep. However with a few exceptions, obstructive sleep apnea is a condition that exclusively affects human beings. Studying animal behavior, we may conclude that most vertebrates in general sleep in a prone position under normal conditions. From an anatomical point of view, sleeping in a prone position is advantageous. A prone position allows for the respiratory tract to remain open during sleep, as the tongue does not tend to fall back into the throat to block the airways, and throat remains open, which is necessary for normal breathing. However, despite this fact, sleeping in a supine or side position is very common among humans. This often results in snoring behavior and to the development of obstructive sleep apnea.

Snoring could be a health problem for the snorer. It may cause sleep fragmentation leading to sleepiness during the day. Snoring is also often a social problem by negatively affecting the sleeping conditions for a partner who shares bedroom with the snorer.

Obstructive sleep apnea is an accompanying phenomenon to snoring, characterized by frequent pauses in breathing leading to reduced oxygen saturation of the blood and arousals during sleep. Similarly to snoring, obstructive sleep apnea often causes daytime sleepiness, and is also recognized as one of the major causes of traffic accidents. Without proper treatment, obstructive sleep apnea can cause an increased risk for cardiovascular diseases, mainly hypertension and cardiac infarction. About 9-15% of the middle age population, mainly men, suffer from OSA.

During the last decades continuous positive airway pressure (CPAP) has come into use as a tool for reducing snoring and obstructive sleep apnea. CPAP is generated by an electromechanical blower, which delivers airflow via a nasal and/or mouth mask with an expiratory resistance. The efficiency of CPAP in reducing the incidence of snoring and sleep apnea is often good when the patient successfully wears it but the compliance and the hours of usage remain relatively low. Numerous studies show less than 50% of compliance one year after introduction of the treatment to the patient. The main problem of CPAP beside the costly equipment and that it is cosmetically unappreciated, is the limited freedom of the user in bed being forced to wear a mask covering nose and/or mouth, and connected via a tube to an electrical apparatus during the sleep. Many people find wearing a mask during sleep unpleasant due to pressure. A cosmetic side effect is also often a remaining swelling under the eyes and pressure marks on the face. Furthermore access to electric mains is necessary which may be a problem in developing countries, but also when travelling, camping and in similar situations.

There are many trials conducted on the usage, efficiency and compliance of CPAP. The efficiency of CPAP in treating OSA is dependent on the pressure produced by the equipment to force the air past the collapsed respiratory airways and into the lungs of the user. Mechanically increasing the pressure will ultimately obtain efficiency up to almost 100%. However the main problem with this treatment is compliance. Due to the low compliance of the equipment, studies conducted on the usage of CPAP consider full compliance to be 20 hours of usage a week (4 hours per night in 5 nights of the week). The statistical results of compliance in usage of CPAP would be much lower calculating with an average of 6-8 hours per night in an every day usage equaling 42-56 hours of usage per week. Even though considering 20 hours of usage per week being equivalent to 100% compliance as in the medical trials, the normal long-term compliance of CPAP lies around 40-60%.

Another common treatment of sleep apnea is the Mandibular Advancing Device (MAD), which is an oral appliance for protrusion of the lower jaw during sleep. Advancing the lower jaw draws the tongue and the soft tissue forward and is aimed at producing more space in the upper airways, which reduces the risk of hypoapneas/apneas during sleep. In conducted medical trials MAD has shown a higher compliance than CPAP, however demonstrating a lower efficiency in treatment of sleep apnea. In addition to discomfort of having a foreign object in the mouth during sleep, MAD may also cause dental and temporomandibular joint problems.

Surgical treatments that have proven efficiency in long-term medical trials are tracheostomy and maxillomandibular advancement surgery. These surgical procedures have substantial side effects for the patients and they are not considered as common alternative treatments for the sleep apnea patients.

Diagnosis of obstructive sleep apnea is made by monitoring several parameters such as oxygen saturation, pulse, thorax and abdominal movements, air flow in and out, snoring and body position during night. Although the diagnostic methods and our knowledge about obstructive sleep apnea have improved considerably during the past years, the treatment of obstructive sleep apnea is still a great challenge for the physician and of considerable problem for the patient. The actual treatment options mentioned above are all together able to help at the most ⅔ of all patients with obstructive sleep apnea leaving ⅓ with no acceptable treatment alternatives. It is mandatory to find new treatment alternatives for patients suffering from obstructive sleep apnea.

It is well known that changing the sleep position from supine to side position can improve snoring and sleep apnea. This will to some extent decrease the tendency of the tongue to fall back into the throat and block the airways.

It is known that obstructive sleep apnea is a disease that almost exclusively affects human beings. Studying the animal behaviour during sleep we can conclude that a main general difference in the sleep habit between humans and other mammals is the sleeping position. While almost all the mammals prefer a prone body and head position, most human beings tend to sleep in a supine or side position. The physical facts supporting the advantage of prone position are the forces of the gravity on the soft tissue in the upper airways. When lying on the back during the sleep with the concomitant muscle relaxation, the soft tissue falls back causing snoring and sleep apnea. Lying on the side normally reduces the snoring and obstructive sleep apnea since the gravitation forces drawing the soft tissue backwards are reduced. With a complete prone head position having the nose perpendicular to the surface beneath the force of gravitation actually helps to pull the soft tissue forward producing more space in the upper airways with the concomitant reduction or elimination of snoring and obstructive sleep apnea. According to this theory, enabling human beings to sleep with the head in a prone position, the obstructive sleep apnea and snoring should be improved or eliminated.

With the above-mentioned theory in mind it is compulsory to differentiate between the different body and head positions during sleep. Hence a prone body position might not necessarily mean a prone head position. It is also important to make a distinction between the different angulations of the head position. A full and true prone head position means having the nose downwards in a perpendicular angle (approx. 90°) to the underlying horizontal surface. A supine head position is the total opposite with the nose pointed upwards in an approx. 90° angle to the underlying horizontal surface. Having the head in the side position is defined as having the nose in an approx. 0° angle pointed approx. parallel to the underlying horizontal surface either to the right or to the left. A semi-prone position is created by having the head with the nose pointing in an approximately 45° angle (or more widely defined as being between 0 and 90° depending on the face contour etc.) to the underlying horizontal surface. Similar angular definitions may be applied to the body position.

The differentiation between these positions is important due to the actual force of gravity on the soft tissue. For instance, having the head in the semi-prone position improves the snoring and sleep apnea but due to the approx. 45° angle the vector of the gravitation force is smaller than in the full prone position with the nose in a downward approx. 90° angle in relation to the underlying surface. In order to optimize the improvement of obstructive sleep apnea the optimal position should therefore be the full prone position with the nose in a 90° angle with the underlying surface for producing the maximal drawing effect of the gravitation, pulling the soft tissue and the mandible forward.

Sleeping with the body in a side or semi-prone position allows for the head being in a side or semi-prone position. However having the head in a true prone position is not possible or is at least very uncomfortable when lying with the body in a side or semi-prone position on a conventional mattress and having a conventional pillow known in the prior art. It is also difficult to have the head in a true prone position when having the body in a prone position supported by conventional mattress and pillow.

A prone body position would be beneficial in order to reach a state of having the head most preferably in a prone position, and thereby reducing snoring and obstructive sleep apnea. However, many people find sleeping with the body in a prone position uncomfortable, and they are not able to sleep for the whole night in this position. There is often a problem of finding a comfortable position of the head and arms, and to move them from one position to another. Moreover, as a result of sleeping with the body in a prone position, the pillow often tends to block the free passage of air to the mouth and nose. The associated experience of heat in the face area and the difficulty in breathing or the sense of resistance of the air flow when breathing is unpleasant and often lead to reduced sleep quality.

Several head and body support systems have been previously described, characterized by pillows and cushions of various materials and in various shapes, intended to support a person in various positions. Some of the previous systems are aimed to support a person lying in a supine position and other systems are adapted for side or prone or semi-prone positions. However none of these previous systems of cushions and pillows have been shown to satisfactorily provide support for the head and the body to sleep in the prone position in order to reduce obstructive sleep apnea. As a consequence no medical trials have previously been published to show the efficiency of prone head and body position in the reduction of sleep apnea.

WO 2009/103109 discloses a solution for reducing snoring and sleep apnea, specifically obstructive sleep apnea. The reduction of these conditions is accomplished by a support device for the support of a person lying in a supine or lateral position. The device comprises a head support arranged to support the head of the user, a rib cage support arranged to support the rib cage of the user, and a recess arranged between the head support and the rib cage support. The recess is adapted to accommodate, at least to some extent, the arm of the user, so as to align the head and spine of the user such that the user's airways are kept open. The degree of flexibility in the choice of the head and arm positions is limited. Furthermore, the head support and the rib cage support are not freely adjustable in relation to each other making it difficult to adjust to individual anatomical features.

U.S. patent publication 2004/0155158 discloses a device supporting the head of the user lying in a prone or side lying position. The device comprises a head support including ventilation means in the form of air channel(s) through the support structure, and a pair of elongated dependent arms that can be manipulated to serve as a chest support. This device allows sleeping not only in a prone position of the body, but also in a side lying position of the body, and it does not prevent the user from moving from a prone position to side or semi-prone position, which is not optimal for reducing snoring and sleep apnea. Furthermore, not providing any substantial support for the body makes it instable to sleep on, and does not allow free exchange of breathing air via the ventilation means having a limited cross-section area determined by the inner walls of the ventilation channel. This application has mostly been used as a support for prone body position lying on the beach for a short period of time and would not be functional on a bed for several hours of usage per night due to the instability and lack of aeration. Some of the positions suggested in the drawings seem strangulating with the risk of asphyxiation of the user at night making this invention hazardous in treating patients.

U.S. Pat. No. 4,987,625 discloses a device comprising a set of cushions that are flexibly and adjustably attached to each other, serving as a head and body support for persons in a supine or prone lying position. The device has chamfered corners on one of the cushions, providing for ergonomic arm support when the user lies in a prone position. Similar to WO 2009/103109, the degree of flexibility in the choice the of head and arm positions is limited, and the head support and the rib cage support are not freely adjustable in relation to each other. Furthermore, the device of U.S. Pat. No. 4,987,625 does not support a sleeping position having a facedown vertically prone position of the head. This device is mostly designed for prone body working position and is not appropriated in the treatment of sleep disorders.

U.S. Pat. No. 6,003,177 discloses a pillow including a pillow body which has a central portion having a first bed head end and a second foot end, a first pair of head support limbs extending outwardly from the central portion and curving from the bed head end towards the foot end, and a second pair of neck, chin and jaw support limbs extending outwardly from the central portion at the foot end of the central portion and being spaced inwardly of the head support limbs. A breathing space is defined between each adjacent head support limb and neck, jaw and chin support limb, and it extends from an intermediate position of the central portion between the bed head end and the foot end, and curving outwardly and towards the foot end of the central portion. The head support limbs and neck, jaw and chin support limbs have surfaces which are curved downwardly from top to bottom of the same so that the breathing spaces taper from a relatively wide opening at the top of the limbs to a relatively narrower opening at the bottom of the limbs. The breathing spaces do not allow free exchange of breathing air due to a restricted cross-section area determined by the sidewalls of the limbs This device does not permit a full prone sleeping position but a side or a semi-prone position during sleep. Specifically it is possible to easily change head position from semi-prone to side during sleep, which is not optimal for reducing snoring and obstructive sleep apnea. It is not possible to have a sleeping position with the head in a prone facedown vertical position. Furthermore, the device does not provide any support for the body and comfortable positioning of the arms, making it troublesome to find a natural anatomical position for the shoulders and the arms. These conditions force the user to change positions in order to avoid numbing due to neurological and circulatory disturbance in the arms in addition to back and neck pain. Insufficient aeration, the impossibility to sleep with a prone head position and the lack of proper body support, gives substantial reduction in efficiency and compliance in treating sleep apnea and snoring for this pillow.

U.S. Pat. No. 4,349,925 discloses a pillow for supporting a person sleeping in a prone body position, but having the head obligatory in a semi-prone position (not true prone position of the head). As it is not symmetric it will allow inclination of the head only in one direction. In addition lacking a proper body and head support makes the pillow rather uncomfortable to lie on with the risk of numbing and pain in addition to the risk for the user to deviate from the intended position.

U.S. Pat. No. 4,118,813 discloses an anti-snoring pillow having a tapered top surface and recesses in the sides of the pillow enabling the mouth and nose to be free, and allowing free access to air for breathing. This is described to be a “training pillow” for the snorer. The pillow according to this patent lacks a proper body support and does not permit a full prone head position, and the recesses are not flexibly adjustable in size. This pillow is designed to teach the patient to avoid the supine sleeping position. For that purpose the pillow might function for a limited period of time. The pillow has a great deal of limitation for actual treatment of obstructive sleep apnea due to its shape and the fact of not permitting the full prone position for the head. The lack of proper body support makes it uncomfortable to use for a longer time and will cause discomfort in the neck, back and the arms.

U.S. Pat. No. 6,226,818 discloses a set of pillows for support of the face of a person lying in a prone position. The firmness of the pillow material is different in different parts of the pillow. An opening is provided between the pillows to provide breathing space for the facedown person. This pillow is the only one in the prior art that intends to provide a full prone position of the head with the nose in a perpendicular angle to the underlying surface. Lacking a body support however, make the pillows uncomfortable and unsteady for the prone head position. The pillows have no support for the forehead, neither for the chin, making them unstable in the inclination of the head and exerting pressure concentrated only to the cheeks making it uncomfortable for usage during a longer period of time. This design might produce pain in the neck and the sensation of sleeping in a tunnel, claustrophobia and asphyxiation due to lack of sufficient aeration beside the problems with the positioning of the arms.

Through the years many pillows having the objective of treating sleep apnea and snoring have been presented. Nevertheless none of these including the presented prior art has been able to show efficiency in the treatment of the disease in conducted medical trials. There are no objective measures supporting the actual function of these pillows due to practical, functional and anatomical imperfections in the design leading to failure in treatment of the patients with obstructive sleep apnea. Therefore, there has been a long felt need for a new and inventive pillow that helps in reducing problems with snoring and/or sleep apnea.

It is an object of the present invention to provide a pillow and mattress set that combines a body support and a head support in an anatomical design adapted for full flexibility in the choice of alternative prone lying positions, allowing a facedown vertically prone position with the nose in approx. 90° to the underlying surface. This will be in combination with free passageways of breathing air to and from the mouth and nostrils. This is important for the user compliance that is required for satisfactory reductions of obstructive sleep apnea and snoring.

It is a further object of the present invention to provide a pillow and mattress set that allows a position of alignment or a relative extension of the neck of the user, which provides widening of the airways. This is achieved by the present invention, but is not achieved by the disclosures according to the prior art.

It is an object of the present invention to provide a pillow and mattress set that allows a true prone position of the head in combination with the possibility of altering the head position between resting on a right hand and a left hand part of the pillow, allowing the side of the face that is supported by the pillow to be shifted between left side and right side, and that also allows a true prone position of the head, having the forehead and not any of the cheeks resting on the pillow. These three alternative head support alternatives by the use of the pillow according to the present disclosure increases the flexibility in use and makes it easier for the user to find various head resting positions without deviating from the true prone head position.

The prone head position reduces obstructive sleep apnea by means of using the force of gravity to pull the mandible, the tongue and the rest of the soft tissue in the throat, responsible of the obstruction, to come towards the jaws and the lips hence producing more space in the hypopharynx of the patient and reducing the symptoms of sleep apnea.

It is a further object to provide a pillow and mattress set to be used as a support for lying/sleeping obligatory with the body in a prone position and the head in a prone position during the whole sleeping session by preventing the user from moving from the prone position while sleeping, for efficient reduction of snoring and sleep apnea syndromes enduringly during the sleeping session. The mattress in the present invention enables the correct anatomical positioning of the elbow and the shoulder joint with a flexion to produce an essentially vertical angle in both of the joints bilaterally but permits free choice of positioning in the extremities.

Furthermore, it is an object of the invention to provide a pillow and mattress set, comprising a pillow to support the head in a prone position and a mattress to support the body in a prone position, wherein the pillow and mattress are freely adjustable in relation to each other in all directions and angles.

Thus it is an object of the invention to provide an improved pillow and mattress set to be used as a means for effectively reducing snoring and obstructive sleep apnea during a complete sleeping session and at the same time offering a comfortable and flexible head and body support that can be adjusted to optimize the lying comfort for optimal sleeping conditions during the whole sleeping session, e.g. the whole night.

It is also an object of the present invention to provide a pillow providing similar benefits as mentioned above for the set of pillow and mattress but not included in a set.

As set forth herein, to effectively construct a head and body support device for reducing snoring and obstructive sleep apnea, three demands should be fulfilled. Firstly, the device should be comfortable and user friendly, providing a pleasant feeling and comfort, which is an important factor for user compliance. Secondly, the device should be so arranged that it prevents moving from a prone body position to a side or supine body position during sleeping in a prone body position, and at the same time allows easy change of head and arm positions. Thirdly, the device should be freely adjustable so as to fit users of different size, weight etc, and users having different preferences when it comes to various prone positions. The different aspects of the invention herein described fulfil these demands. The subject matter of the present invention provides an unexpected and inventive advantage in terms of reduction of snoring and obstructive sleep apnea compared to prior art. None of the above mentioned inventions and prior art, neither separately nor in combination, describes the present invention.

### SUMMARY OF THE INVENTION

It is an object of the invention to provide an improved pillow and mattress set to be used as a means for reducing snoring and obstructive sleep apnea during sleep by allowing a true prone position of the head.

It is a further object to provide a pillow and mattress set as a support for lying with the body in a prone position for reduction of snoring and obstructive sleep apnea syndromes during sleep.

A further object of the invention is to provide a pillow and mattress set, comprising a pillow to support the head in a prone position and a mattress to support the body in a prone position, wherein the pillow and mattress are freely adjustable in relation to each other.

In an embodiment of the invention the pillow has a generally T-shaped design in top view, comprising a bar portion with two opposite ends and a protrusion portion extending from approximately the midpoint of the bar portion, and the protrusion portion and the bar portion define corners and corner spaces, which corner spaces are open to the surrounding air in at least the direction opposite to the corners.

In another embodiment, the protrusion portion is widening from the bar portion outwards.

In still another embodiment the protrusion portion is narrowing from the bar portion outwards.

In another embodiment the bar portion is widening towards the ends.

In another embodiment the bar portion is curved towards the corner spaces.

Further in another embodiment the top surface and the bottom surface of the pillow are parallel to each other.

In yet another embodiment the top surface of the pillow is inclined towards the protrusion portion in relation to the bottom surface.

In another embodiment an indentation is provided opposite to the protrusion.

In another embodiment the pillow is provided with a formable but essentially non-elastic strip incorporated in the bar and protrusion portions, and having an orientation essentially the same as the orientation of said bar and protrusion portions, the strip enabling bending and molding of the pillow into desired shapes that persist until bent again.

In one embodiment of the mattress said mattress is provided with recesses at each side of one of the short ends.

In another embodiment said mattress is provided with recesses at each side of both short ends.

In yet another embodiment said mattress is provided with a recess at one of the short ends for supporting the chin of a user.

In another embodiment the mattress is thicker at one of the short ends compared to the other short end.

In one embodiment said pillow and said mattress are separate units, so they can be moved spatially and placed in various orientations without limitation in relation to each other and in relation to a user.

In another embodiment the set further comprises one or several base sections that can be placed and positioned as desired under the mattress to allow a user to locally alter the height and the support of the user of the mattress.

In another embodiment the pillow and/or the mattress further comprise a pressure, movement and/or temperature sensor and/or a microphone, for monitoring and registration of frequency and/or duration of use by a user and the frequency and duration of snoring and apneas by the user.

In another embodiment the pillow and/or the mattress further comprise inflatable segmented air chambers enabling height adjustment of each segment of the mattress and/or pillow by the degree of inflation.

Other objects and advantages of the present invention will become obvious to the reader and it is intended that these objects and advantages are within the scope of the present invention.

### BRIEF DESCRIPTION OF THE DRAWINGS

FIG. 1 shows perspective views of an exemplary embodiment of the pillow according to the present invention.

FIGS. 2 and 3 are two different perspective views of the set of pillow and mattress according to an exemplary embodiment of the invention. The set comprises a pillow designed to support the head of a person and a mattress designed to support at least part of the body (chest, shoulders and abdomen) or the whole body of said person, when the set of pillow and mattress is used as a support by said person when lying in a prone position.

FIGS. 4a-l show top views, and FIGS. 5a-e show side views of different alternative embodiments of the pillow of the invention. FIG. 5a shows a typical head position on the pillow in side view.

FIGS. 6a-i show top views, and FIGS. 7a-d show side views of different alternative embodiments of the mattress of the invention. FIG. 7b shows a slight inclination in the mattress at side view and FIGS. 7c and d show various embodiments having a thickening towards one of the short ends of the mattress. All the mattresses in FIGS. 6 and 7 can be provided in full length (to support the length of the whole body except the head) or can be shorter to support part of the body of the user.

FIGS. 8a-f are cross-sectional views of different alternative embodiments of the pillow and the mattress wherein various materials or properties of materials are indicated by various patterning which is described more detailed elsewhere in this specification.

FIGS. 8g and h are cross-sectional views of different alternative embodiments of the mattress, and FIGS. 8i and 8j are the corresponding top views, wherein base sections (FIGS. 8g and 8i), or individually inflatable sections in the base portion of the mattress (FIGS. 8h and 8j), for adjustment of thickness of various parts of the mattress are indicated by various patterning.

FIGS. 9a-c are top views of exemplary ways of positioning the pillow in relation to the head. The position of the head is indicated.

FIGS. 10a-d show various alternatives of positioning of the pillow and the mattress and various positions for the user's arms. FIG. 10d also shows an alternative embodiment of the mattress.

FIGS. 11a-c show perspective views of the pillow and mattress set with various alternative positioning of the user.

FIGS. 12a-d and FIG. 13 show an alternative arrangement for the control of position of the pillow in relation to the mattress and a bed. FIG. 14 shows an alternative embodiment of the mattress to enable the conversion of the mattress of the present invention into an ordinary mattress and vice versa.

### DETAILED DESCRIPTION OF THE INVENTION AND PREFERRED EMBODIMENTS THEREOF

The pillow and mattress set of the invention will now be described in detail with reference to the figures. The figures are exemplary embodiments of the invention, and the invention is not restricted to these general embodiments, but will also include alternative designs and dimensions as will be described elsewhere in this application, and will also include altered dimensions and shapes and choice of materials that are obvious to the man skilled in the art.

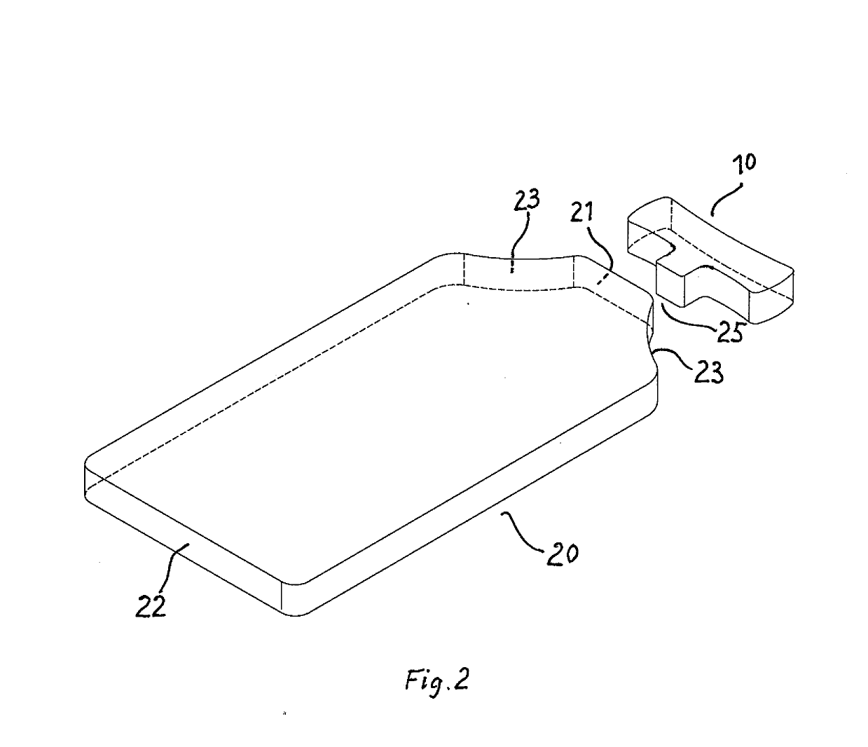

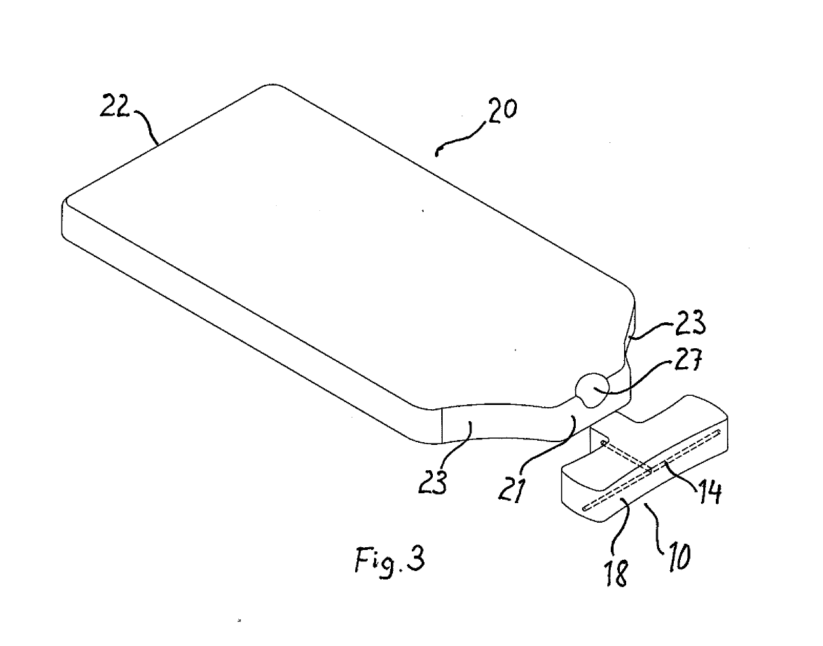

In FIGS. 2 and 3, the orientation of the pillow and mattress in relation to each other, and in relation to the user when in use, is one of several preferred orientations. The orientation according to FIG. 2 will be referred to in this application as a standard orientation only for sake of simplicity in the description of the pillow and mattress as it makes it possible to define the different parts of the pillow and mattress in relation to the specified standard orientation. The upwardly facing surfaces of the mattress and pillow in FIG. 2 are defined as top surfaces, and the opposite surfaces are defined as bottom surfaces. Other orientations of the pillow and mattress in relation to each other and in relation to the user are preferred as well, e.g. the pillow turned around approximately 90° , 180° or any angle in relation to the mattress. The pillow may also be elevated in relation to the mattress.

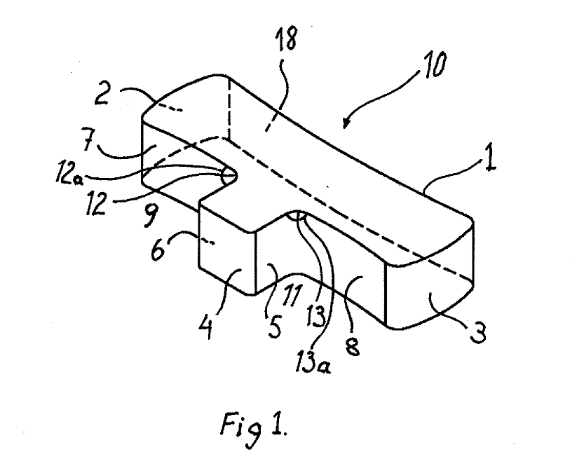

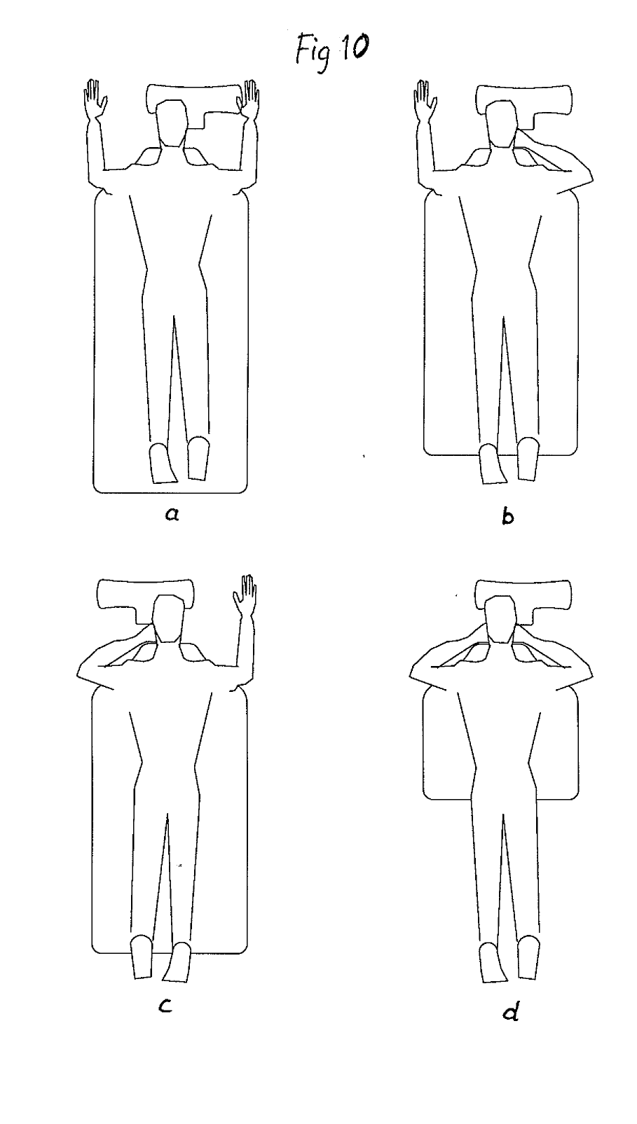

Having said that, with reference to FIG. 1 demonstrating a preferred embodiment of the invention, the pillow 10 is characterized by having a generally T-shaped design as viewed from the top, comprising a bar portion 1 with two opposite ends 2, 3 which are left-hand and right-hand oriented, respectively, in relation to the user, and having one protrusion portion 4 extending perpendicularly to said ends from approximately the midpoint of the bar portion and towards the mattress in standard orientation, i.e. towards the foot end of the user in standard orientation. Each side 5, 6 of said protrusion portion 4 and the correspondingly opposite sides of the perpendicular left-hand 7 and right-hand 8 sides of said bar 1 define corners 12, 13 and corner spaces 9 and 11. Each of said corner spaces 9, 11 is open to the surrounding air in at least the direction opposite to the corners 12, 13, and the spaces 9, 11 widen up from the corners outwards, wherein the widening depends on the corner angle 12a, 13a. The outwardly widening corner spaces 9, 11 provide excellent breathing air passageways in any prone position of the head resting on the pillow, having the head in full prone position with the nose pointed downwards vertically to the underlying surface (FIGS. 5a, 9a-c and 11a-c).

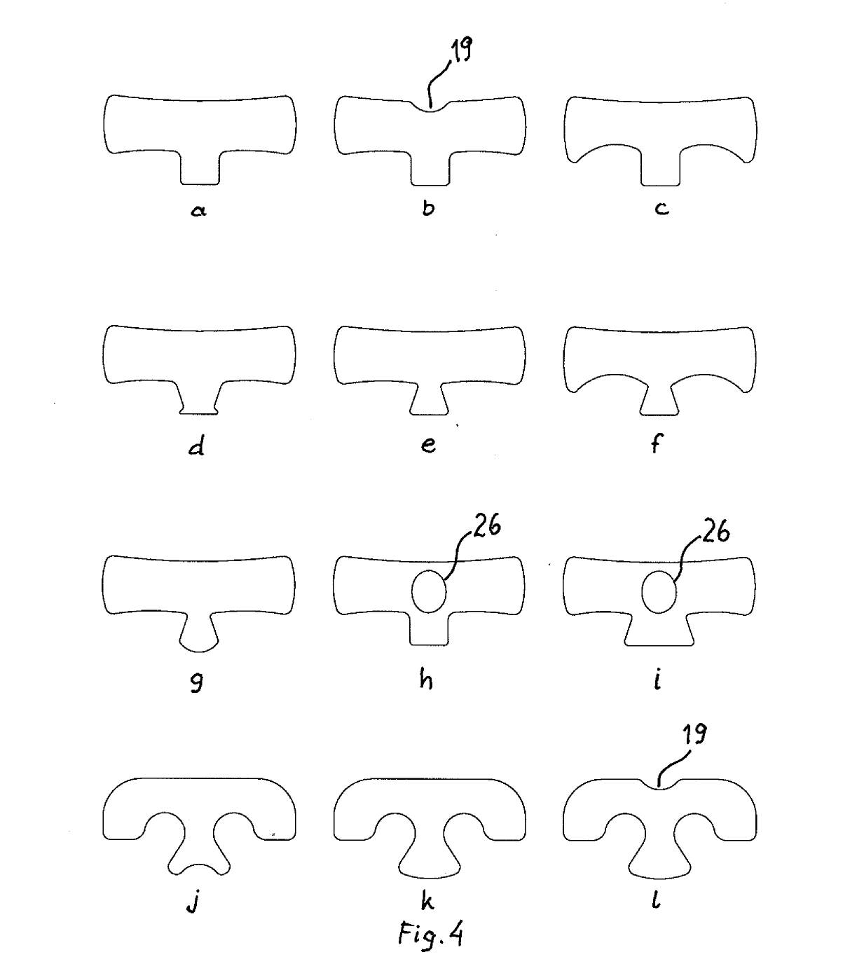

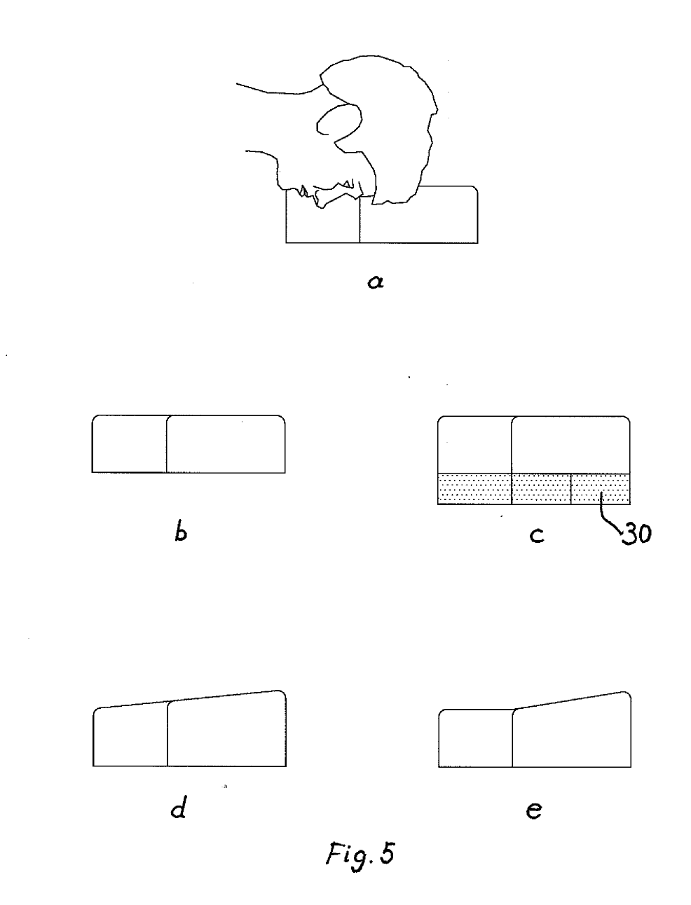

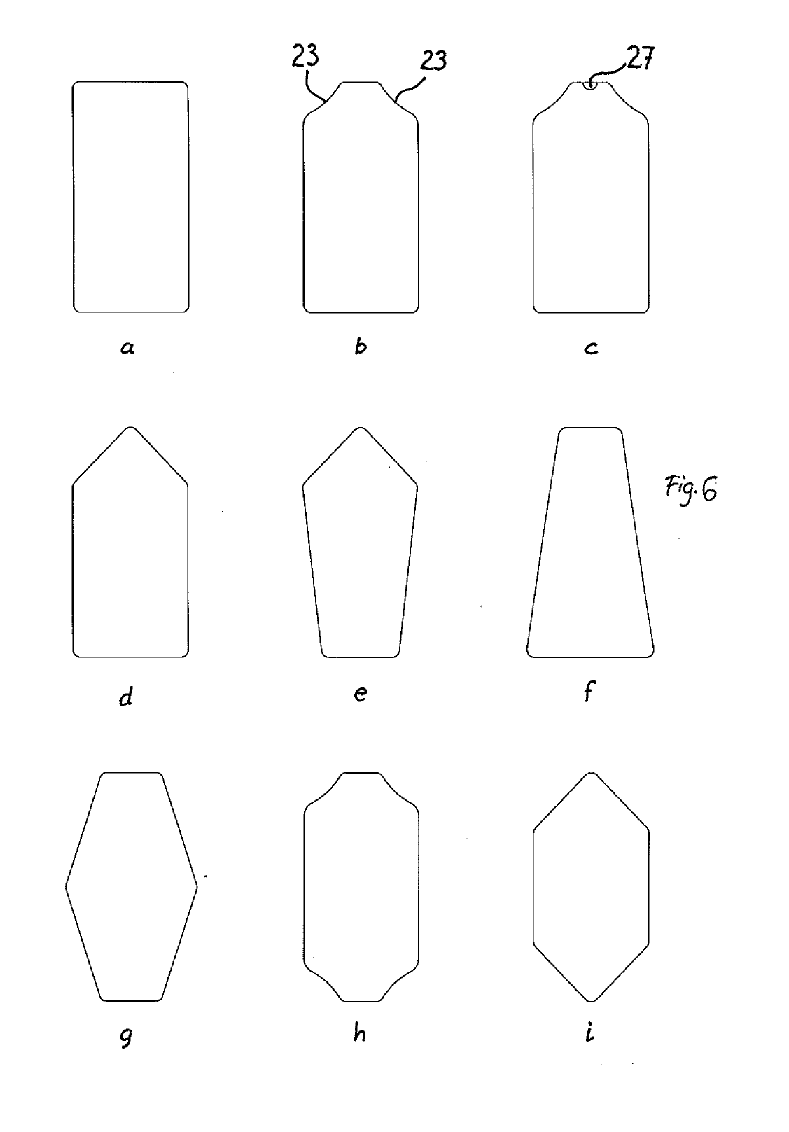

FIG. 4 shows examples of various embodiments of the pillow 10. FIG. 4b represents the exemplary embodiment of FIG. 1. FIGS. 4d-4l represent alternative configurations of the protrusion 4. FIGS. 4c, 4f and 4j-4l show embodiments with alternative configurations of the bar portion 1. As can be seen from these figures, the bar portion 1 is advantageously widening towards the ends 2,3 (FIG. 4c, 4f), or is curved towards the corner spaces 9, 11 (FIG. 4j-4l). Such curvatures may be a preformed feature of the pillow or may be a result of bending a formable strip 14 described elsewhere. FIGS. 4h and 4i show an alternative embodiment of the pillow 10 with an opening 26 in the central part of the pillow 10 to provide less weight of the pillow and improve the anatomical positioning of the head and aeration.

In an alternative embodiment of the pillow 10, the protrusion 4 extending towards the mattress in standard orientation is widening from the bar outwards, i.e. towards the mattress as viewed from top (e.g. FIGS. 4e-4g and 4i-4l) or is narrowing from the bar outwards, i.e. towards the mattress (FIG. 4d). The corner angles 12a, 13a in FIG. 1 and the rate of widening of the associated corner spaces, will depend on the rate of narrowing or widening of the protrusion 4.

FIG. 4b represents an embodiment of the bar portion 1 having an indentation 19 as will be described more in detail here after.

As is apparent from FIG. 4, the term “corner” in broad sense refers to the point or zone 12, 13 where the sides of the bar portion (7 and 8) and the protrusion portion (5 and 6) meet. In some of the embodiments presented there are no obvious corner angles as said zones are wide (FIG. 4j-l)

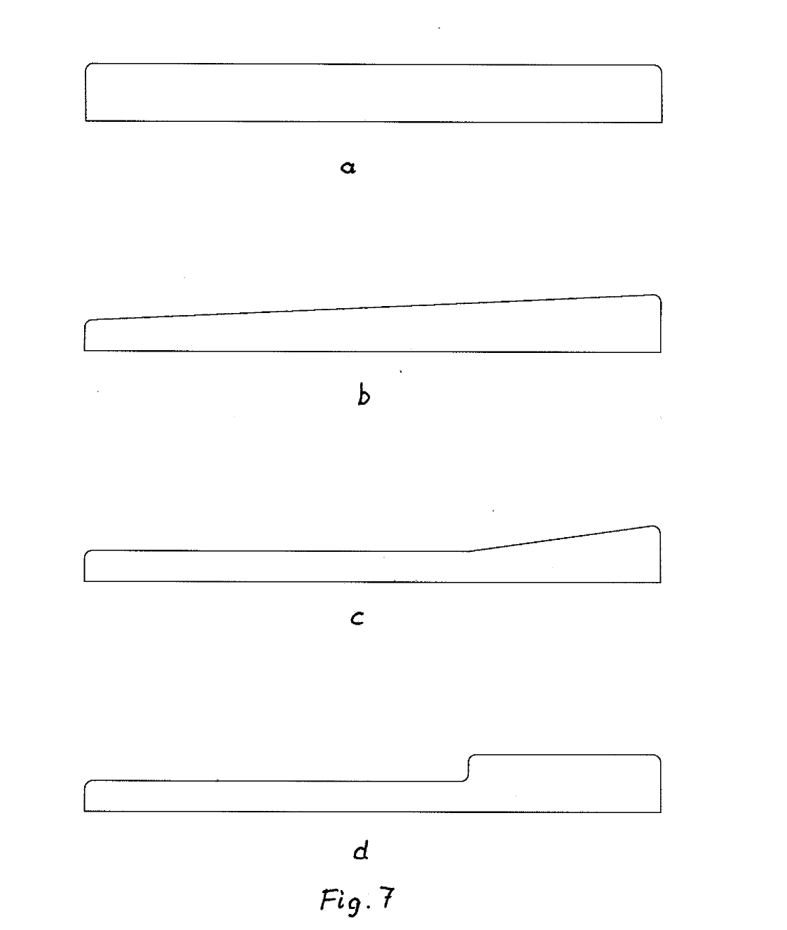

FIGS. 5a-c represent the pillow having a top surface that is horizontal (FIGS. 5a-c) or alternatively is partly (FIG. 5e) or entirely (FIG. 5d) inclined. Referring to FIG. 2, the mattress 20 is characterized by having a generally rectangular design with excavations 23 or recesses 23 (both terms are used interchangeable throughout this patent application) for the arms as viewed from the top. The opposite short ends 21, 22 of the mattress are preferably oriented towards the pillow, i.e. towards the head end of the user, and towards the foot end of the user, respectively. The recesses 23 are situated at each side of one of the short ends as indicated in FIG. 2. In an alternative embodiment there are recesses at each side of both short ends of the mattress, enabling positioning of both arms and both legs in relation to the recesses as indicated in FIGS. 6g-i.

FIG. 3 illustrates the present invention in an alternative perspective, and shows also an alternative embodiment of the mattress, with a recess or excavation 27 (both terms are used interchangeable throughout this patent application) that can be used for comfortable resting of the chin of the user. FIG. 3 also shows a formable strip 14 incorporated in the pillow body 10, which enables bending and molding of the pillow into desired shapes that persist until bent again and that will fit the head of the user.

FIG. 6 shows various configurations of the mattress. FIGS. 6b and 6c represent the preferred embodiment provided with excavations or recesses 23 for positioning of arms. In FIG. 6c the recess 27 for supporting the user's chin is also indicated. FIG. 6d-f represent alternative embodiments of the mattress.

FIGS. 6g-i represent various alternative configurations of the mattress permitting the positioning of the arms and legs/feet in the excavations (recesses) provided respectively at the head and foot ends of the mattress. All the body mattresses can be provided in various lengths for full or partial body support.

FIGS. 7a to 7d represent various alternative embodiments of the mattress having different thicknesses, with (FIG. 7b) or without (FIG. 7a) an inclination of the top surface and with thickening of the top of the mattress towards one of the short ends (FIGS. 7c and 7d). In the latter cases the mattress is thicker at one of the short ends compared to the other short end.

FIGS. 9-11 show different positioning alternatives for the head, arms, legs and the body on the pillow and mattress set. The user can choose whether to have or not have the chin, the throat and the feet resting of the mattress as illustrated.

The mattress having different and varying thicknesses, different length/extension supporting only the chest/shoulders, or also the abdomen and legs are envisaged as well. Depending on the height, the size and personal preferences of the subject using the mattress, it can be provided in different length and width to cover a small part or the most part of the bed as illustrated in FIG. 10.

The mattress and pillow of the invention are advantageously placed upon a standard bed mattress. In an alternative embodiment, the pillow and mattress of the invention are placed directly on a bottom of a bed, e.g. a bed stead, having the ordinary mattress of the bed removed.

The pillow 10 can also be provided on its own (not included in a set of pillow and mattress). In this embodiment the pillow may be used as described in this specification, preferably in combination with any suitable mattress having dimensions as desired.

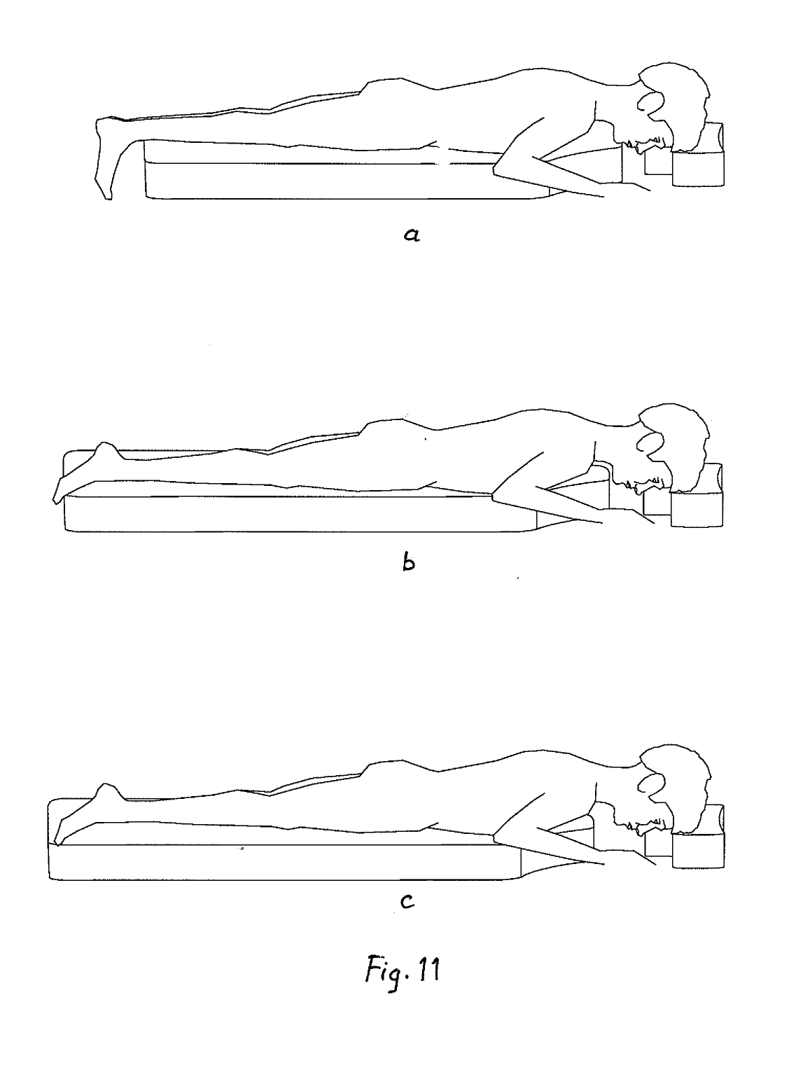

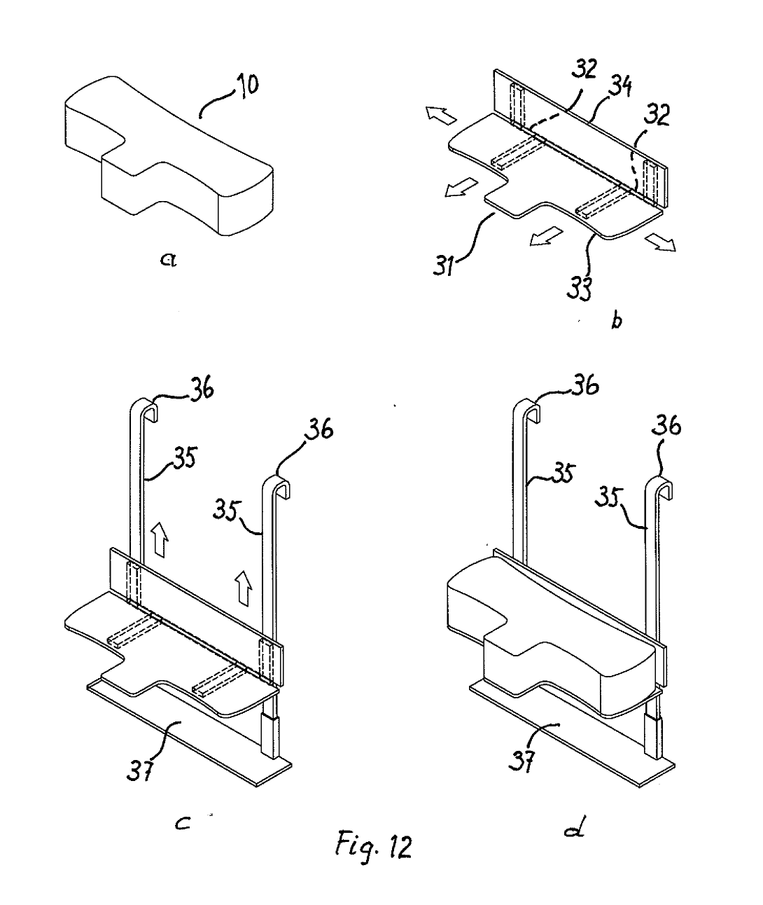

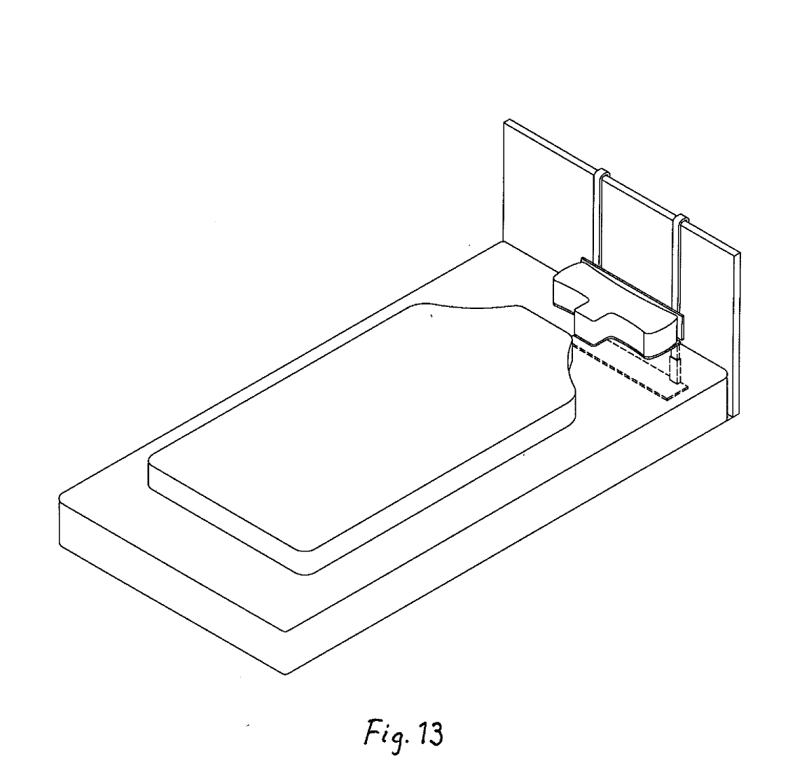

The set of pillow and mattress according to this invention provides a comfortable support for the head and the body, optionally including chest, shoulders, neck, forehead, chin, cheek, legs, feet and abdomen, of a user lying in a prone position, depending on the length, size, design and orientation of the pillow and mattress set and the free choice of the patient. In standard orientation the set of pillow and mattress preferably supports the head in a facedown vertical prone position with support for at least the forehead, one of the cheeks on the pillow when the protrusion 4 is orientated towards the mattress (FIG. 2, 3, 10, 11) and with (FIG. 11a) or without (FIGS. 11b and 11c) supporting the chin and throat on the mattress depending on the medical preferences and the patient's choice.

Furthermore, the set of pillow and mattress is preferably designed to freely allow natural movement of the neck and head during sleep, enabling the person to change between various alternative true prone positions of the head during the sleeping session, while preventing movement from a prone position to a side or semi-prone position of the body.

The set of pillow and mattress according to this invention allows a person lying on it in a prone body position with free movement of the arms and legs with the choice of position of the head with left or right hand support although always in a prone head position. FIGS. 10a-d show alternative positioning of the arms and the shoulders in relation to the mattress and the pillow. Other positions are also envisaged.

FIG. 14 shows an alternative embodiment, which makes it possible to convert the mattress in the present invention into an ordinary mattress for the non-snorer (indicated by arrows). The mattress can easily be reconverted into an antiapnea mattress with simple removal the upper part 28 (indicated by arrows).

The pillow and mattress of the set according to the invention are not fixed or mechanically coupled to each other, and therefore can be moved spatially without limitation in relation to each other and in relation to the user. Also, without limitation, they can be placed in various orientations in relation to each other and in relation to the user.

As presented in FIG. 2 the pillow and mattress set is advantageously placed with some distance 25 between the head pillow and the body mattress in order to provide air passage from both sides. The width of this distance 25 is free of choice and may be zero.

The pillow 10 is designed to support the head of the user lying in a prone position. Depending on how the pillow is oriented in relation to the mattress 20, different areas of the head of the user lying in a vertical prone position with or without neck extension (depending on the height of the pillow in relation to the mattress) can be supported, i.e. forehead (FIG. 9c) or cheek, side of head and forehead (FIGS. 9a-b). A preferred position of the pillow 10 is with the protrusion 4 directed towards the mattress 20 (FIG. 2). In this position the subject can lay in a true prone head position with the nose perpendicular to the underlying horizontal surface, resting the forehead and either left or right side of the face (left or right cheek) on the protrusion 4 of the pillow (FIGS. 5a, 9a-b, 10, 11). The pillow 10 is typically positioned slightly towards the left or slightly towards the right in relation to the mattress, in order to allow the user to rest his/her face in a prone position and resting one of his/her cheeks on the protrusion and rest the body on the mattress as shown in FIG. 10. Having the pillow oriented to the left allows the user to rest his/her left cheek on the protrusion, and having the pillow oriented to the right allows the user to rest his/her right cheek on the protrusion (FIG. 10).

An alternative use is to turn the head pillow 10 around approx. 180° and rest the forehead on the bar portion 1 as shown in FIG. 9c. To make the side 18 of the bar more comfortable to the head, an indentation 19 can be provided as represented by FIGS. 4b and 6c.

The excavations or recesses 23 on each side of the mattress 20 make it possible for the user to place one or both arms towards the head end giving an anatomically comfortable position for the arms. These excavations or recesses 23 are also helpful in avoiding deviation from the desired prone position on the mattress. Similar excavations or recesses for both arms and legs/feet are provided in FIG. 6h. The correct anatomical positioning in the shoulder and the elbow require a semi-flexed or approximately perpendicular angle in these joints. These angles are impossible to achieve with the prior art. The mattress raises the body of the user relatively to the bed permitting a natural flexion in the shoulder joint as envisaged in FIG. 11. Furthermore a flexion in the elbow joint will be natural, positioning the arms forward (FIGS. 10 and 11). Other positions of the arms and shoulders are also possible in the set of pillow and mattress and described the FIGS. 10 and 11.

The corner spaces 9, 11 provide a breathing opening or environment for the nose/mouth of the user in a true prone head position of the head of the user when resting or during the course of sleep in a substantially prone body position. In addition to these corner spaces 9 and 11 there is also typically a space 25 between the pillow and the mattress, which further improves airflow and oxygenation and reduces heat production and discomfort.

The top surfaces of the pillow and mattress can provide a smooth skin and body contact surface area in any position during sleep and also minimizes the wrinkling by pressure markings of the face skin during sleep. The soft material of the pillow and mattress provides a comfortable surface to sleep on and due to the impression of the body parts it is difficult to deviate from the desired prone position.

In the most preferred embodiment the top material is compressive in the preferred viscoelastic “memory foam” material in order to permit adaptation to the weight and the body contours of the user, nevertheless other materials with different properties and hardness are also envisaged as described below.

In the preferred embodiment the mattress consists of a soft surfacing viscoelastic material permitting moulding of the body on it's surface. The impressions divide the exerted pressure evenly on the body and the head of the user making the mattress and pillow highly flexible and comfortable. Furthermore the impressions from different body parts form a framework that impedes deviation from the prone position. The hardness of the mattress and pillow set is preferably adapted depending on the weight and other anatomical features of the user.

In conclusion, the set of pillow and mattress provides flexibility of ease of adjustment during the night. Whenever needed the pillow or alternatively the mattress may be repositioned easily as desired for personal and medical preference. The shape of the set makes it possible for the user to find a sleep position as desired without deviating from the intended prone position. By prone is meant a position of the head having the nose essentially directed downwardly, in an essentially vertical direction in relation to the essentially horizontal surface of the bed. Some medically negligible degree of inclination from the vertical direction is meant to be included in the meaning of a prone head position.

In an alternative embodiment the pillow 10 and/or the mattress 20 of the invention has/have a pressure, movement and/or temperature sensor and/or a microphone incorporated in it, for monitoring and registration of frequency and/or duration of use and the frequency and duration of snoring and apneas. The user may have a positioning sensor attached to his/her body or clothes, e.g. attached to a belt around his/her waist like a belt. The pressure or temperature sensor can provide detailed information of use, e.g. how long period of time the user lies in a prone position and in a supine position, respectively, during a sleeping session.

Preferably the pillow and mattress have individual covers conforming in shape to each body of the pillow and mattress, respectively.

In an alternative embodiment the side 18 of the pillow 10 which is opposite to the foot end directed protrusion 4 may also be provided with an indentation 19, FIGS. 4b, 9. This come into function as a space for the eyes and adjusted to the shape of the head of the user in a face-down prone position when resting or during the course of sleep having the pillow turned around approx. 180° from the standard orientation (FIG. 9c).

Referring to FIGS. 5d and 5e, in an alternative embodiment the top surface of the pillow is slightly inclined to offer an alternative positioning of the head, with the neck extended, which position is known to increase the cross section of the air ways in the throat, and thereby being helpful in reducing OSA. The inclination may cover the entire top surface (FIG. 5d) or a part of the top surface (FIG. 5e).

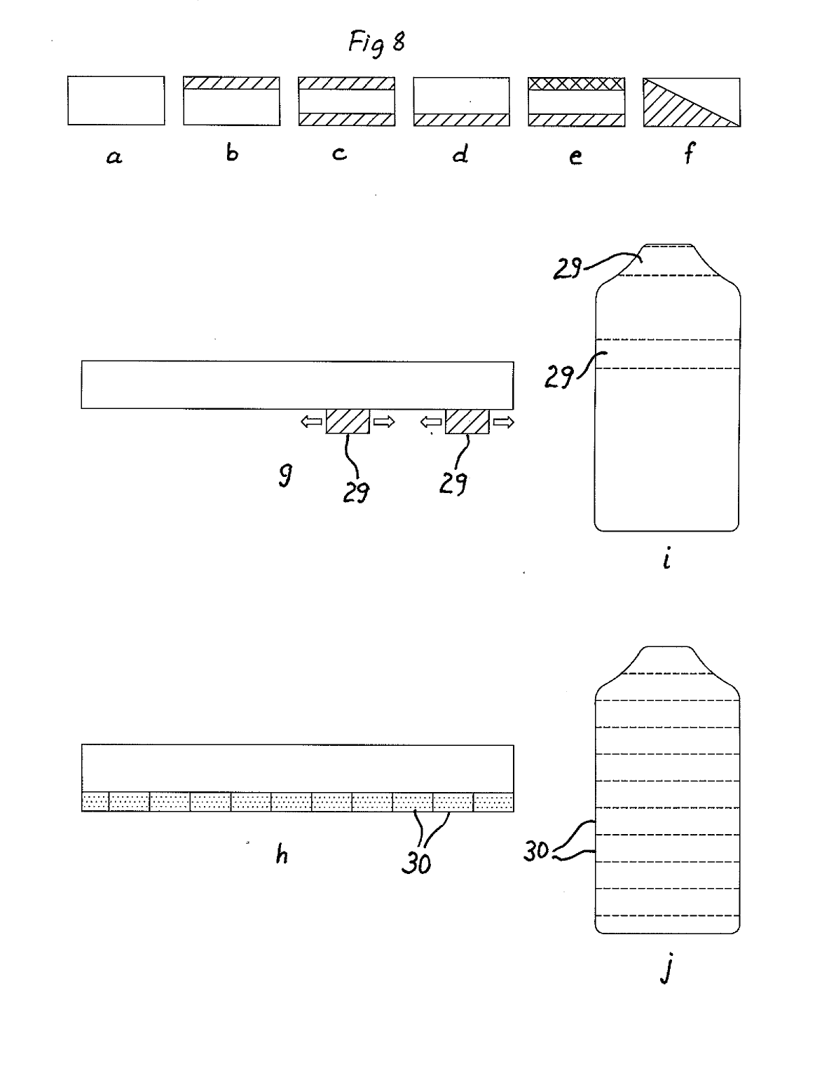

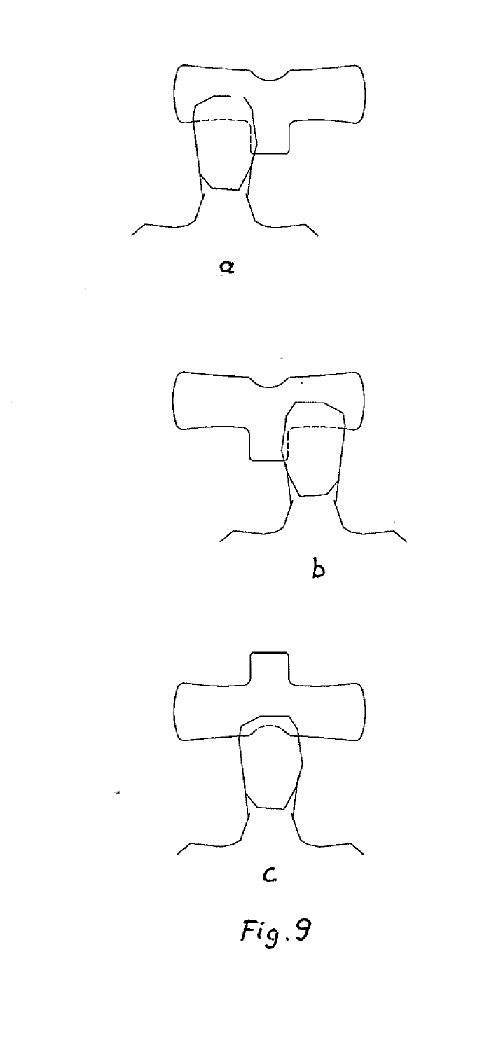

The fact of providing two separate units (pillow and mattress) makes it possible to adjust the positioning but also the height of the pillow in relation to the mattress in all directions and angles in order to achieve the proper positioning of the head and the body of the user. An alignment or sometimes a relative extension in the neck may for instance be achieved also by choosing a higher pillow compared with the mattress. As shown in the FIGS. 8, 12 and 13 an alteration in height of the mattress and pillow for adjustment to personal anatomical features and weight of the user is envisaged by several mechanisms. To obtain a comfortable prone sleeping position it is imperative to have a relaxed angle in all the joints, especially in the neck. The amount of pressure on different body parts such as hips, breasts and stomach are dependent of the user's anatomical features. To achieve the correct angle in the joints and a controlled and adjustable pressure to each region independently of individual anatomical features it is mandatory to provide a mechanism for adjustment of different parts of the pillow and mattress, As shown in FIG. 8g a simple measure to raise the level of the mattress is demonstrated by placing foam material 29 below specific regions where special support is needed. In FIG. 8h mattress with individual air chambers 30 is placed below the main mattress and pillow (5c), which enables the raising or lowering of each individual segments of each entity to adapt to the users weight and body contour. These individual air chambers may be inflated manually or by mechanic or electric pumps connected to each chamber. Several mechanisms are envisaged to provide attachment and stability between the air chambers and the main pillow and mattress e.g. hooks, button, glue, belts, and releasable fasteners sold under the trademark Velcro.

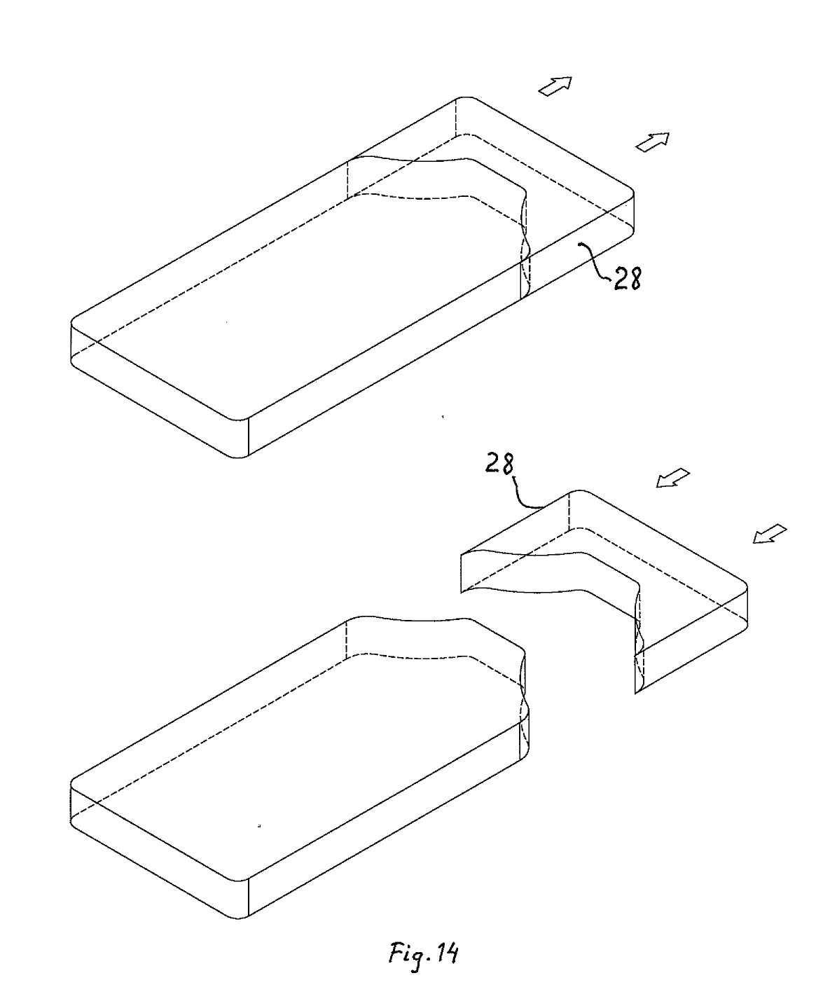

In an alternative embodiment the pillow may be mechanically raised and lowered to achieve a comfortable sleeping angle and provide more aeration during respiration. As described in FIGS. 12 and 13, a structure resembling a shelf (31) is provided for placement of the pillow 10. The shelf structure (31) consists of a horizontal 33 and a vertical 34 plate that are united by joints 32, which permit protrusion, retrusion, lateral movements in addition to angulation of the horizontal plate 33 in relation to the vertical plate 34. The vertical plate is positioned upon rails 35, which permit upward and downward replacement of the shelf structure. The shelf is fixated in the horizontal plane and movement and positioning is whether enabled by upward tilting of the shelf more than a certain degree level, by a release button or a combination of these mechanisms.

The rails 35 may be provided preferably in a rigid or mouldable but preferably a non-elastic material. Alternative materials are metal, wood, plastic, leather and textile but other materials or combination of the above mentioned materials are envisaged. The upper ends of the rails 36 have a semi-circular or a hook shape in order to fit on most types of headboards on the beds. The lower part 37 consists of a stabilizing support which is adjustable in height. When this arrangement is in position the upper angulated part 36 is preferably positioned to be attached to the upper part of the headboard of the bed and the lower part 37 is preferably placed beneath the bed and adjusted in height to be stable and rigid. In an alternative embodiment the rails 35 are attached to a vertical surface, which is attached to the headboard of the bed or the adjacent wall (in case of bed with no head board).

As presented in FIG. 3, in a preferred embodiment of the invention the pillow 10 is provided with formable but essentially non-elastic T-formed strip 14 incorporated in the bar and protrusion portions, and having an orientation essentially the same as the orientation of the bar and protrusion portions (the strip indicated in dashed lines in FIG. 3). The strip is preferably made of metal or plastic coated metal or any material giving the strip the desired formable properties. Bending the strip to alter its form results in the bar and/or protrusion portion being similarly altered in its form as well. By bending the strip and bar/protrusion, the user can alter the form of the pillow as desired adjusting it to the size of the head, the shape of the cheeks and the position of the eyes. Examples of alternative bendings of the bar is demonstrated in FIG. 4j-l.

The size of the pillow 10 and mattress 20 of the invention shall be such that they fit the size and body configuration of the user. Some non-limiting examples of the dimensions of the pillow and mattress are given here. A typical dimension of the pillow might be around 20-60 cm from one side to the opposite side in any direction. The width of the protrusion 4 in a typical embodiment is approximately ⅕-⅓ of the total width of the pillow. A typical length of the protrusion 4 is approximately ⅓-¼ of the total length of the pillow. A typical dimension for the mattress might be around 60 to 200 cm in length and 60 to 120 cm in width. These are just very approximate examples of dimensions to give the reader an idea of the appearance of the invention, and the exact dimensions of a preferred set of pillow and mattress are not limited to these figures and depend on the body shape and weight of the user and are adjusted to the size of the bed. The relative measures of the pillow and mattress in relation to each other appear in general terms from the figures. Also the dimensions of the different parts of the pillow and mattress set appear from the figures in general. The figures are examples of preferred embodiments and a preferred set of pillow and mattress is not limited to the relative measures presented in these figures.

Referring to FIG. 8a-f, the pillow and mattress preferably include one or several layers composed of breathing elastic/flexible material such as foam rubber, foam plastic, preferably a viscoelastic foam or plastic material such as memory foam or silicone, or composed of fibre, down, feather, hollow fibre or similar stuffing and also compartments with air or water giving a flexible pillow/mattress. If the pillow or mattress comprises several layers, the different layers may be composed of different materials or material blends, represented by the variously patterned layers of FIG. 8a-f with or without inclinations relative to each other. In one of the preferred embodiments of the invention the pillow and/or mattress comprise(s) more than one layer of distinctive physical properties, FIG. 8b-f. In an exemplary embodiment, a top layer having a higher compressibility and a bottom layer having a lower compressibility can be provided for increased comfort and anatomical adaptation to the body in combination with appropriate stiffness and rigidity of the pillow/mattress, FIGS. 8b, 8d. In another exemplary embodiment, three layers with different physical properties are provided, FIG. 8c. The various layers can be of various thicknesses and can vary within the mattress or pillow body as represented schematically by FIG. 8.

As shown in FIG. 8g in one embodiment of the mattress one or several separate base sections 29, can be placed and positioned as desired to allow the user to locally alter the height and the support of the mattress. These separate base sections 29 are arranged at the bottom side of the mattress according to the invention as is represented by FIGS. 8g and 8i.

The pillow 10 and/or mattress 20 may also in an alternative embodiment entirely or partly be inflatable or be filled with water or another liquid similar to waterbeds. As shown in FIGS. 8h, 8j and 5c the lower part of the mattress and the pillow can consist of inflatable segmented air chambers 30, which enable height adjustment of each segment of the mattress and pillow for height, pressure and compressibility depending of the anatomical, medical and personal preferences of the user.

In an alternative embodiment additional soft filling may be provided for location between the pillow/mattress body and the outer cover for changing the height and/or contour of the pillow or mattress. Different surfacing materials are envisaged in order to provide the desirable “surface feeling” for the user. The surfacing material enhances the comfort by reducing the heat production, sweating and the feeling of pressure and gives a sensation of freshness and relaxation. The surfacing materials can be provided as integrated in the mattress or in the sheeting to cover the mattress and pillow set. Yet another “surface feeling” is provided by movable particles within the mattress and pillow to give an alternating pressure sensation in the different parts of the body even though the user is lying in the same position.

In a preferred embodiment the thickness of the pillow is around 5-40 cm. A more preferred thickness of the pillow is 12-25 cm. In a preferable embodiment the thickness of the mattress is around 5-40 cm. A more preferred thickness of the mattress is 15-25 cm. In a preferred embodiment the pillow is somewhat thinner than the mattress. The preferred thickness partly depends on the material characteristics and the users anatomical features.

The pillow and mattress can be manufactured by various techniques and of a variety of different materials. A preferred mode of producing the pillow and mattress is by cutting or punching the intended profile from a standard sheet of raw material of the desired thickness and including the desired foam material or including the desired layers of different materials with the desired properties regarding stiffness, elasticity, flexibility, colour etc. Another preferred method to produce the pillow and mattress is by press moulding or injection moulding, using moulds of desired dimensions for the production of the pillow and mattress. An advantage of the latter method is that pillows and mattresses of various thicknesses in different parts of the individual pillow or mattress can be produced.

In the alternative embodiment of pillow and mattress made inflatable with air or for filling with water or other liquid, production of the pillow and mattress may be by welding plastic-coated fabric or the like. The inflatable pillow-mattress set may provide a practical alternative for travellers suffering from sleep apnea.

In the alternative embodiment of pillow and mattress made of a cover filled with down, feather, fibre, hollow fibre etc. or other stuffing, the production may be by sewing a cover and filling the cover with the stuffing material.

The mode of producing the pillow and mattress according to the invention is not restricted to the methods here described. Any procedure resulting in a pillow and mattress having the properties described in this specification is within the scope of the present invention.

### EXAMPLES

### Example 1

A sheet like foam plastic raw material was provided, the material having a thickness of 15 cm for the mattress and 12 cm for the pillow. The foam plastic had a compressibility comparable to a standard foam plastic mattress. A pillow and mattress set according to the invention herein, that is a first head support pillow and a second body support mattress, was manufactured by cutting out from the raw material the desired forms of the pillow and mattress. As viewed from the top, the pillow had a shape of a “T” as in FIG. 2a, and the mattress was rectangular with excavations for the arms, as represented by FIG. 3b. The pillow was 12 cm and the mattress 15 cm thick in an uncompressed state. The pillow measured 45 cm in width (the length of the bar), and 30 cm in the perpendicular direction thereto, the protrusion direction, the protrusion being 12 cm long and 10 cm wide. The essentially rectangular mattress measured 80×160 cm with excavations 23 for the positioning of the arms as in FIG. 3b. The pillow and mattress set was covered with lined cotton cloth, for comfort and hygiene.

The pillow and mattress were placed on top of a 90×210 cm standard mattress upon a regular bedstead. The pillow and mattress were arranged according to the standard orientation of FIG. 1, having the pillow on the head end of the bed and the mattress approximately at the mid-area of the bed.

For an experimental setup, three persons suffering from severe snoring and obstructive sleep apnea, diagnosed by sleep monitoring at previous occasions were invited to sleep on the pillow and mattress set while monitoring the sleep quality of each subject. These patients experienced failure of treatment with conventional therapy (CPAP and MAD) and were invited to try the new invention.

The subjects were advised to lie down on a pillow-mattress set according to this invention and were instructed to sleep on it in a prone position. While asleep several parameters such as oxygen saturation, pulse, thorax and abdominal movements, air flow in and out, snoring and body and head position during night were monitored and compared with results from previous sleeping sessions without the pillow and mattress of this invention. The conditions, like time of the day, temperature, degree of silence and light, were equal to the previously monitored sleeping sessions, which were performed at most 4 weeks earlier for the persons in question.

All the test subjects showed improvement in the severity of the snoring and the frequency of apneas during sleep, with at least 50% reduction in apneas compared the previous measurements made at the same sleep laboratory. No negative side effects whatsoever were registered with use of the invention herein.

### Example 2

A study including 9 patients with obstructive sleep apnea showed promising results in hours of usage per night, comfort, and most important in reduction of the tendency of obstructive sleep apnea. Successful treatment of obstructive sleep apnea in previous studies is generally defined as the reduction of the number of apneas by at least 50%. In the conducted medical trial on the described pillow and mattress set presented here, it was shown that: a) 2 patients couldn't adapt to the prone sleeping position and could not complete the study; b) 7 patients completed the study; c) the study patients slept in average 80% of the night in the prone position: d) 6 of 7 patients reduced the tendency of sleep apnea with more than 50% with average reduction of 68%; e) all the 7 patients following the treatment had reduced snoring; and f) 1 of 7 patients showed no reduction in the apnea tendency.

| | | | Nr of obstructive | Nr of | Percentage of |
|---|---|---|---|---|---|
| | | | apneas per hour of | apneas per hour of | reduction of |
| | | | sleep without the | sleep with the | obstructive sleep |
| Patient | Sex | Age | pillow and mattress | pillow and mattress | apnea per hour |
| 1 | male | 39 | 19 | 5 | 74% |
| 2 | female | 58 | 91 | 42 | 54% |
| 3 | male | 68 | 22 | 6 | 73% |
| 4 | male | 38 | 38 | 7 | 82% |
| 5 | male | 48 | 28 | 12 | 57% |
| 6 | male | 38 | 22 | 21 | 0.5% |
| 7 | male | 34 | 61 | 18 | 70% |

1. A pillow for use as a head support, especially for support of the head of a human in a prone position during sleep, the pillow having top and bottom surfaces,
wherein the pillow (10) has a generally T-shaped design in top view, comprising a bar portion (1) with two opposite ends (2, 3) and a protrusion portion (4) extending from approximately the midpoint of the bar portion (1), and the protrusion portion (4) and the bar portion (1) define corners (12, 13) and corner spaces (9, 11), which corner spaces (9, 11) are open to the surrounding air in at least the direction opposite to the corners (12, 13).

2. The pillow according to claim 1, wherein the protrusion portion (4) widens from the bar portion (1) outwards.

3. The pillow according to claim 1, wherein the protrusion portion (4) narrows from the bar portion (1) outwards.

4. The pillow according to claim 1, wherein the bar portion (1) widens towards the ends (2, 3).

5. The pillow according to claim 1, wherein the bar portion (1) curves towards the corner spaces (9, 11).

6. The pillow according to claim 1, wherein the top surface and the bottom surface of the pillow are parallel to each other.

7. The pillow according to claim 1, wherein the top surface of the pillow is inclined towards the protrusion portion (4) in relation to the bottom surface.

8. The pillow according to claim 1, wherein an indentation (19) is provided opposite to the protrusion (4).

9. The pillow according to claim 1, wherein the pillow (10) is provided with a formable but essentially non-elastic strip (14) incorporated in the bar (1) and protrusion (4) portions, and having an orientation essentially the same as the orientation of said bar and protrusion portions, the strip enabling bending and molding of the pillow into desired shapes that persist until bent again.

10. A mattress (20) for use as a body support in combination with the pillow (10) according to claim 1, especially for support of a human body in a prone position during sleep, said mattress having top and bottom surfaces and two short ends (21, 22), wherein said mattress (20) is provided with recesses (23) at each side of one of the short ends (21).

11. The mattress according to claim 10, wherein said mattress (20) is provided with recesses (23) at each side of both short ends (21, 22).

12. The mattress according to claim 10, wherein said mattress (20) is provided with a recess (27) at one of the short ends (21) for supporting the chin of a user.

13. The mattress according to claim 10, wherein the mattress (20) is thicker at one of the short ends (21) compared to the other short end (22).

14. A set comprising a pillow (10) according to claim 1, and a mattress (20) for use as a body support, especially for support of a human body in a prone position during sleep, said mattress having top and bottom surfaces and two short ends (21, 22), and having recesses (23) at each side of one of the short ends (21) wherein said pillow (10) and said mattress (20) are separate units, so they can be moved spatially and placed in various orientations without limitation in relation to each other and in relation to a user.

15. The set according to claim 14, wherein the set further comprises one or several base sections (29) that can be placed and positioned as desired under the mattress (20) to allow a user to locally alter the height and the support of the user of the mattress (20).

16. The set according to claim 14, wherein the pillow (10) and/or the mattress (20) further comprise a pressure, movement and/or temperature sensor and/or a microphone, for monitoring and registration of frequency and/or duration of use by a user and the frequency and duration of snoring and apneas by the user.

17. The set according to claim 14, wherein the pillow (10) and/or the mattress (20) further comprises inflatable segmented air chambers (30) enabling height adjustment of each segment of the mattress and/or pillow by the degree of inflation.

18. A method for repositioning of the pillow (10) according to claim 1, the method comprising the steps of providing a structure resembling a shelf (31) for placement of the pillow (10) upon the shelf (31), which structure resembling a shelf (31) comprises a horizontal plate (33) and a vertical plate (34) united by joints (32) for protrusion, retrusion, lateral movement and angulation of the horizontal plate (33) in relation to the vertical plate (34), wherein the vertical plate (34) is positioned upon rails (35), permitting upward and downward replacement of the structure resembling a shelf (31) along the rails, placing the pillow on the shelf and repositioning the pillow upward and downward along the rails, and by protrusion, retrusion, lateral movement and/or angulation of the horizontal plate by means of the joints (32).
